(19) United States
(12) Patent Application Publication
(10) Pub. No.: US 2008/0015595 A1
(43) Pub. Date: Jan. 17, 2008

(54) Screw-Comprising Two Threads With Different Pitches, Used to Join Two Bone Segments

(75) Inventors: Xavier Renard, Vauhallan (FR); Michel Merle, Luxembourg (FR)

Correspondence Address: YOUNG & THOMPSON, 745 SOUTH 23RD STREET, 2ND FLOOR, ARLINGTON, VA, 22202, US

(73) Assignee: XAVIER RENARD, VAUHALLAN (FR)

(21) Appl. No.: 10/558,646

(22) PCT Filed: May 17, 2004
(86) PCT No.: PCT/FR04/01205

§ 371 Date: Nov. 13, 2006

(30) Foreign Application Priority Data
May 30, 2003 (FR) 03/06559

(51) Int. Cl. A61B 17/58

(52) U.S. Cl. 606/73; 606/60

(57) ABSTRACT

Self-compressing screws, especially self-threading screws, include a screw shaft having an axis of revolution, the screw shaft being provided with two threads that are centered on the axis of revolution, and a first envelope in the form of a rotary hollow cylinder defined by the thread, between the base and the tip of an individual thread section, fitting entirely inside the second envelope in the form of a rotary hollow cylinder defined by the thread, between the base and the tip of an individual thread section. A pitch P₁₁ of the thread is smaller than a pitch P₂₂ of the thread. A method of applying the screw for joining two bone segments is also provided in order to perform an osteosynthsis operation between the two bone segments.

### FIELD OF THE INVENTION

The present invention relates to self-compressive screws, and preferably self-tapping screws, to unite two bone portions for the production of an osteosynthesis between these two bone portions.

### BACKGROUND OF THE INVENTION

There exist devices permitting performing osteosynthesis between two bone portions, for example after fracture of this bone or to eliminate the cause of a malady in the articulation between two bones, etc.

Among numerous devices which are used to obtain such a result, there exist quite simply bone screws. The screw shank is screwed into a first of the two bone portions, to pass through it, and then to penetrate partially the second bone portion, and a nut or the like is screwed on the end of the portion of the shank which emerges from the first bone portion.

Such a technique gives good results overall but has several drawbacks, particularly in the case of uniting small bones such as those of the hand or other bones of the same type.

The principal drawback resides in the fact that, in the case of small bones, it is very difficult or even impossible to retract the portion of the screw which emerges with the nut, with the risk of wounding the flesh surrounding the portion of bone to be united.

There also exists a self-compressive screw system such as the one described in U.S. Pat. No. 6,030,162. However, to obtain self-compression with this system, it is necessary that the screw threads be conical, and that it comprise moreover three screw threads as in the embodiments shown in FIGS. 1, 3, 8, 9, 17 to 22 of this document, or two threads but with moreover a nut numbered 140 in the embodiments shown in FIGS. 11 to 16 of this same document.

### SUMMARY OF THE INVENTION

The present invention thus has for its object to provide a self-compressive and self-tapping screw to unite two bone portions so as to obtain osteosynthesis between these two bone portions, which largely overcomes the drawback mentioned above of bone screws of the prior art, having a relatively simple structure minimizing the number of constituent elements relative to the screws of the prior art, whilst permitting obtaining a very good self-compression.

More precisely, the present invention has for its object a self-compressive screw to unite two bone portions so as to perform osteosynthesis between these two bone portions, comprising a screw shank defined on an axis of rotation, and two first and second screw threads provided on said screw shank, these two first and second screw threads being centered on said axis of rotation, characterized by the fact that the first envelope in the form of a hollow cylinder of rotation defined by the first screw thread between the bottom and tip of its screw thread is entirely comprised within the second envelope in the form of a hollow cylinder of rotation defined by the second screw thread between the bottom and the tip of its screw thread, and that the pitch of the first thread is smaller than the pitch of the second thread.

### BRIEF DESCRIPTION OF THE DRAWINGS

Other characteristics and advantages of the invention will become apparent from the following description, given with respect to the accompanying drawings by way of illustration but in no way limiting, in which:

The three FIGS. 1-3 are views in longitudinal half cross-section according to a conventional representation well known in industrial design, of a member of rotation or substantially of rotation, in which:

FIG. 1 showing the principle of a self-compressive screw according to the invention,

FIG. 2 showing a view permitting understanding the operation of the self-compressive screw according to the invention, and

FIG. 3 schematically showing a preferred embodiment of the self-compressive screw according to the invention.

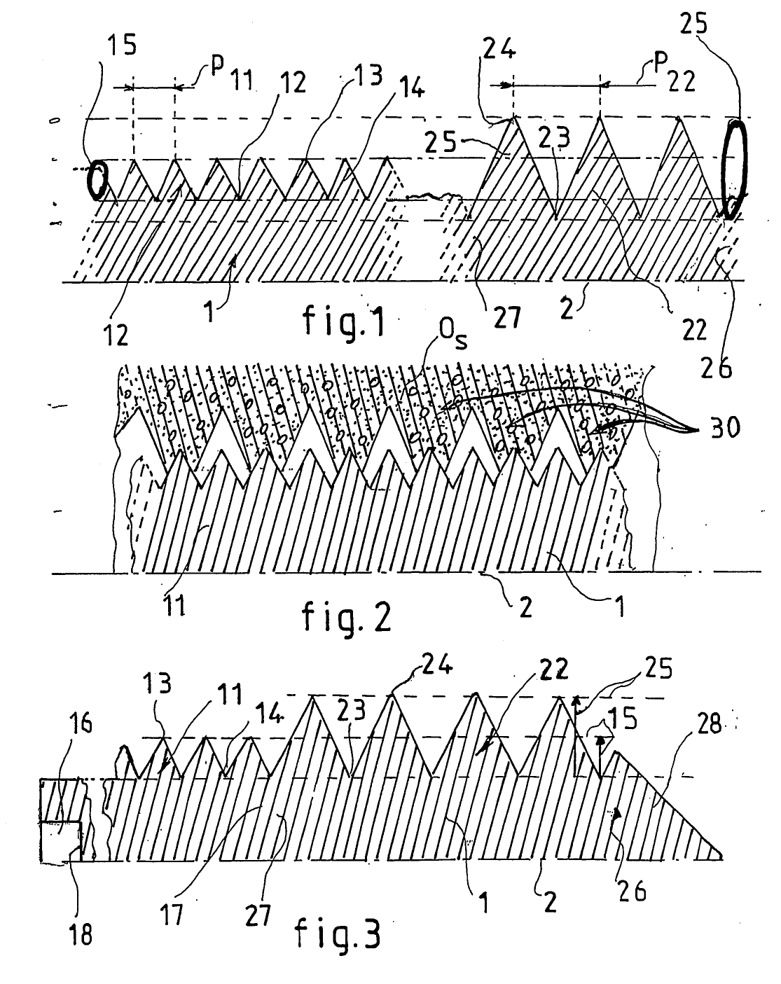

### DETAILED DESCRIPTION

With reference to the accompanying drawings, the screw according to the invention relates to a self-compressive screw to unite two bone portions Oₛ for the performance of osteosynthesis between these two bone portions.

This screw comprises a screw shank 1 designed on an axis of rotation 2, and two first and second screw threads 11, 22 provided on the screw shank 1, these two first and second screw threads being centered on the axis of rotation 2.

According to one characteristic of the invention, the first envelope 15 in the form of a hollow cylinder of rotation centered on the axis 2 and defined by the first screw thread 11 between the bottom 12 and the tip 13 of its screw thread 14 is entirely comprised within the second envelope 25 which is also in the form of a hollow cylinder of rotation centered on the axis 2 and defined by the second screw thread 22 between the bottom 23 and the tip 24 of its screw thread 25.

Moreover, the pitch P₁₁ of the first screw thread 11 is smaller than the pitch P₂₂ of the second screw thread 22.

It should be noted that the two screw threads 11, 22 provided on the shank 1 can be consecutive without a space separating them, but that they could also be separated for example by a smooth portion of the shank.

In one preferred industrial embodiment, as is shown in FIG. 3, the internal diameter of the first envelope 15 is substantially equal to the internal diameter of the second envelope 25.

According to an embodiment which has given very good results for osteosynthesis on bones of the hand, the difference between the external diameter and the internal diameter of the first envelope 15 is substantially equal to half the difference between the external diameter and internal diameter of the second envelope 25, and the pitch P₁₁ of the first screw thread 11 is substantially equal to half the pitch P₂₂ of the second screw thread 22.

These screws being used in the bone field, as mentioned above, it is preferable, to avoid the production of pilot holes for their placement, that the end 26 of the second screw thread 22 opposite that 27 which is turned toward the first screw thread 11, be shaped as a self-tapping penetrating end 28.

As to the end 16 of the first screw thread 11 opposite that 17 which is turned toward the second screw thread 22, it is shaped with an end 18 for gripping by a screwing tool. This end 16 can comprise, as shown, a slot, a polygonal recess, etc., with which the tool can coact such as a screwdriver or the like without it being necessary to provide a shouldered head as for conventional screws. Such a screwdriver is a well-known auxiliary in the field of bone repair surgery.

Preferably, the shank of the screw shank 1 and the two first and second screw threads 11, 22 are made from a single piece or of biocompatible material such as titanium or a titanium alloy.

The self-compressive screw described above operates in the following manner, with reference more particularly to FIG. 2:

First of all, the two bone portions between which osteosynthesis is to be effected, are held against each other by any temporary means, well known in the field of reparative bone surgery.

Then, the screw according to the invention is screwed through first of all the first bone portion, by tapping thanks to the end 28 of the second screw thread 22. FIG. 2 shows the screw thread 30 obtained in the first bone portion Oₛ after the passage of the second screw thread 22.

After having passed through the first bone portion Oₛ, the second screw thread 22 of the screw penetrates the second bone portion and, by self-tapping, is also screwed in. Simultaneously, because of its structural characteristics defined above, the first screw thread 11 screws into the screw thread 30 formed with the second screw thread in the first bone portion, as shown in FIG. 2.

However, as the first screw thread 11 has a pitch less than that of the second screw thread 22, there is produced a relative retractive movement of the two bone portions relative to the self-compressive screw, the retractive movement of the second bone portion being of greater amplitude than that of the first bone portion because, structurally, the pitch P₂₂ is greater than the pitch P₁₁. The difference of relative retraction of the two bone portions is in fact proportional to the difference of the values of the two pitches P₁₁ and P₂₂.

As a result, the two bone portions come into contact with each other, if they were not already.

By exerting a supplemental screwing force on the screw, it is even possible to exert a supplemental compressive force on the two bone portions against each other, to promote osteosynthesis.

The screwing of the screw takes place by the gripping head 18, as mentioned above.

The screw according to the invention has other advantages than those mentioned above. Particularly, it does not require screwing of a nut on the end of the screw shank emerging from the first bone portion. After the screw has been screwed into the two bone portions and the compressive force has been applied by the practitioner, this emerging portion of the screw shank 1 can be cut off flush with the first bone portion, without a portion extending dangerously into the surrounding flesh.

It should also be emphasized that, after the screw has been cut, the practitioner can himself apply supplemental screwing to totally penetrate into the first bone portion the end which has undergone cutting, so as completely to retract it and to be certain that no portion of the screw protrudes dangerously into the surrounding flesh.

Moreover, when osteosynthesis is performed and obtained, it is possible that the screw will no longer be necessary and that the practitioner can withdraw it by unscrewing.

To do this, the screw according to the invention can preferably comprise screwing-unscrewing means such that it can be easily subjected to supplemental screwing after its emerging end has been cut off or be unscrewed after having been implanted.

These screwing-unscrewing means to obtain the objects mentioned above, can be constituted by at least one longitudinal flap parallel to the axis of the screw shank produced on the first screw thread 11, this flap being adapted to provide a grip for pliers or the like to drive the screw in rotation in one direction or the other as needed.

1. Self-compressive screw to unite two bone portions (Oₛ) for the performance of osteosynthesis between these two bone portions, comprising a screw shank defined on an axis of rotation, and two first and second screw threads provided on said screw shank, these two first and second screw threads being centered on said axis of rotation, wherein the first envelope in the form of a hollow cylinder of rotation and defined by the first screw thread between the bottom and the tip of its screw thread is entirely comprised within the second envelope in the form of a hollow cylinder of rotation and defined by the second screw thread between the bottom and the tip of its screw thread, and the pitch (P₁₁) of the first screw thread is smaller than the pitch (P₂₂) of the second screw thread.

2. Screw according to claim 1, wherein the internal diameter of the first envelope is substantially equal to the internal diameter of the second envelope.

3. Screw according to claim 2, wherein the difference between the external diameter and the internal diameter of the first envelope is substantially equal to half the difference between the external diameter and the internal diameter of the second envelope.

4. Screw according to claim 1, wherein the pitch (P₁₁) of the first screw thread is substantially equal to half the pitch (P₂₂) of the second screw thread.

5. Screw according to claim 1, wherein the end of the second screw thread opposite that which is turned toward the first screw thread is shaped as a self-tapping penetrating end.

6. Screw according to claim 1, wherein the end of the first screw thread opposite that which is turned toward the second screw thread is shaped as an end for gripping by a screwing tool.

7. Screw according to claim 1, wherein the screw shank and the two first and second screw threads are made in a single piece of a biocompatible material.

8. Screw according to claim 1, wherein said biocompatible material is titanium or titanium alloy.

9. Screw according to claim 1, wherein it moreover comprises screwing-unscrewing means.

10. Screw according to claim 9, wherein said screwing-unscrewing means are constituted by a longitudinal flap parallel to the axis of the screw shank provided on the first screw thread.
